(19) United States
(12) Patent Application Publication
(10) Pub. No.: US 2025/0352819 A1
(43) Pub. Date: Nov. 20, 2025

(54) BRACHYTHERAPY DEVICE

(71) Applicants: Cornell University, Ithaca, NY (US); Mission-Driven Tech, Inc., Hampton Bays, NY (US)

(72) Inventors: Eve McDavid, Ithaca, NY (US); Onyinye Balogun, Ithaca, IN (US)

(21) Appl. No.: 18/869,259

(22) PCT Filed: May 25, 2023
(86) PCT No.: PCT/US23/23629

§ 371 Date: Nov. 25, 2024

Related U.S. Application Data
(60) Provisional application No. 63/346,110, filed on May 26, 2022.

(51) Int. Cl. A61N 5/10

(52) U.S. Cl. CPC A61N 5/1016; A61N 2005/1018

(57) ABSTRACT

A brachytherapy device including a primary guide, a telescoping primary applicator, a locator assembly for fixing the primary guide and the telescoping applicator, and a control system for managing the different components of the device is described. The primary applicator can be adjusted based on patient anatomy. The locator assembly includes a secondary applicator and a secondary guide connected to an expandable structure that can be inflated after insertion into the patient.

### PRIORITY CLAIM

This application claims priority to U.S. Provisional Application Ser. No. 63/346,110, filed 26 May 2022, which is expressly incorporated by reference herein.

### BACKGROUND

Brachytherapy, also known as internal radiation, is an important procedure that is a part of the standard of care in the definitive treatment of Stage IB-IVA cervical cancer, medically inoperable endometrial cancer, and other gynecological cancers. Each year, thousands of women undergoing brachytherapy endure, on average, five procedures over a three-week period, almost all of whom report extreme pain, distress, and/or post-traumatic stress disorder as a result of post-procedure device removal. Because of this, many patients do not complete the treatment, despite knowing that it will improve outcomes for them.

Some devices used to administer internal radiation for brachytherapy are known as “Tandem and Ring” and “Tandem and Ovoid” applicators. These applicators were developed in the 1970s before women were included in clinical trials and have designs that trace to Marie Curie's discovery of radium. Using these applicators can produce a host of challenges and complications for physicians and patients alike, especially in treating cervical cancer and other gynecological cancers.

As a result of the rigid, inflexible device design, a patient can be less able to tolerate the pain associated with the procedure, even with sedation during insertion. She will sometimes require more extensive anesthesia and pain management for device placement, incurring unnecessary side effects and increased costs. While insertion occurs under sedation in the US, removal occurs while patients are awake and very much aware of the pain and fear associated with removing rigid, inflexible devices after she's just tolerated intensive radiation treatment. The majority of women liken the intense pain to childbirth and academic research has documented women expressing their preference to die from cervical cancer instead of continuing with further treatment of this type. Research has also shown that the painful removal is highly traumatic.

Thus, there is an urgent need to develop a device that efficiently delivers this life-saving treatment to patients by causing minimal pain. Brachytherapy devices in line with the present disclosure provide a tool designed to fit each woman's anatomy so physicians can focus on administering care as opposed to stitching solutions together for treatment.

### SUMMARY

The present disclosure is directed to a brachytherapy device adapted to improve fit, facilitate device adjustment after insertion, and avoid physical damage during and after procedures. In particular, the disclosed brachytherapy device is adapted for use in the treatment of cervical cancer and other gynecological cancers.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a partially diagrammatic view of a brachytherapy device, showing the device inserted into a patient to temporarily expose the patient to radioactive material as part of a brachytherapy procedure;

FIG. 2 is a side elevation view of the brachytherapy device including a telescoping applicator with an extension component in an extended position showing that the extension is configured to be driven to a customized arcuate shape when in the extended position by guide wires;

FIG. 3 is a side elevation view of the brachytherapy device including a telescoping applicator with an extension component in a retracted/contracted position;

FIG. 4 is an illustration of the brachytherapy device with a primary guide and a locator assembly integrated into the device that is configured to substantially fix the device in place during a procedure;

FIG. 5 is an illustration of a control system configured to manage different components of the brachytherapy device of FIG. 4;

FIGS. 6A-6D illustrate inflation of an expandable structure positioned in the locator assembly of the brachytherapy device of FIG. 4;

FIG. 7 is a first embodiment of is expandable structure positioned in the locator assembly of the brachytherapy device of FIG. 4;

FIG. 8 is a second embodiment of is expandable structure positioned in the locator assembly of the brachytherapy device of FIG. 4;

FIG. 9 is a third embodiment of is expandable structure positioned in the locator assembly of the brachytherapy device of FIG. 4;

FIG. 10 illustrates a change in angle of the extension component after insertion of the brachytherapy device of FIG. 4 into the patient;

FIG. 11 illustrates a change in length of the extension component after insertion of the brachytherapy device of FIG. 4 into the patient;

FIG. 12 illustrates a lateral movement of secondary guides of the locator assembly away from the primary guide after insertion of the brachytherapy device of FIG. 4 into the patient;

FIG. 13 illustrates inflation of a first expandable structure of the locator assembly after insertion of the brachytherapy device of FIG. 4 into the patient;

FIG. 14 illustrates inflation of a second expandable structure of the locator assembly after insertion of the brachytherapy device of FIG. 4 into the patient;

FIG. 15 illustrates a configuration of the brachytherapy device of FIG. 4 used to provide radioactive therapy to the patient;

FIG. 16 illustrates deflation of the second expandable structure of the locator assembly prior to removal of the brachytherapy device of FIG. 4 from the patient;

FIG. 17 illustrates deflation of the first expandable structure of the locator assembly prior to removal of the brachytherapy device of FIG. 4 from the patient;

FIG. 18 illustrates a lateral movement of secondary guides of the locator assembly toward the primary guide prior to removal of the brachytherapy device of FIG. 4 from the patient;

FIG. 19 illustrates a change in length of the extension component prior to removal of the brachytherapy device of FIG. 4 from the patient;

FIG. 20 illustrates a change in angle of the extension component prior to removal of the brachytherapy device of FIG. 4 from the patient;

FIG. 21 illustrates a kit comprising expandable structures with varying maximum diameters;

FIG. 22 illustrates an embodiment of an expandable structure that can be locked at varying diameters depending on patient-specific intrauterine anatomy; and

FIG. 23 is a flow chart illustrating a method of using the brachytherapy device for providing radioactive therapy to the patient.

### DETAILED DESCRIPTION

A brachytherapy device for intrauterine procedures and methods of use are disclosed herein. A brachytherapy device according to the present disclosure is configured for administration of intrauterine radiation to treat cancer.

The brachytherapy device 10 disclosed includes a primary guide 12, a telescoping primary applicator 14, a locator assembly 30 for fixing the primary guide 12 and the telescoping primary applicator 14 in place during a procedure, and a control system 50 for managing the different components of the device 10 as shown in FIG. 4. In some embodiments, the brachytherapy device 10 may include only the primary guide 12 and the telescoping primary applicator 14. In other embodiments, the brachytherapy device 10 may include only the locator assembly 30.

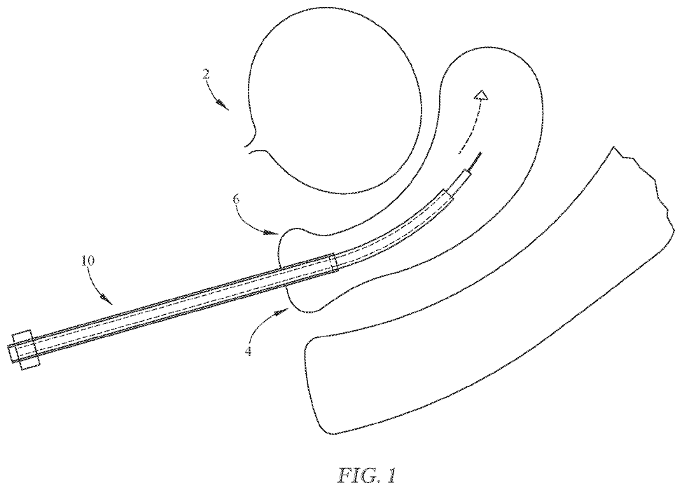

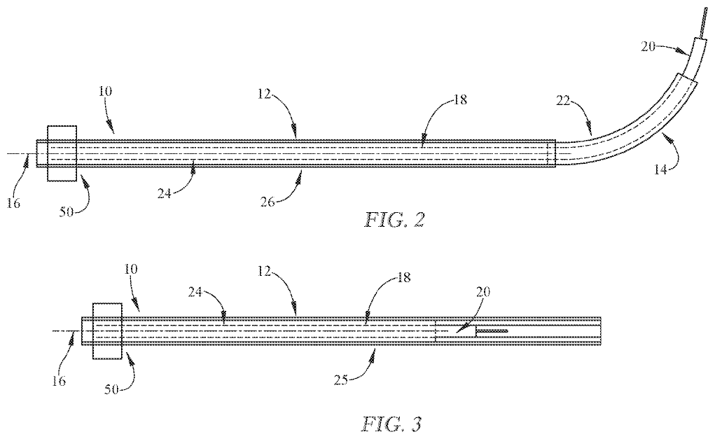

As shown in FIG. 2, the primary guide 12 extends along a primary guide axis 16 and that defines a primary interior space 18 along the primary guide axis 16. In the illustrated example, the primary guide 12 is a hollow tube 12. The primary telescoping applicator 14 is configured to house a radioactive material or a radioactive source 20 within a patient 2 (shown in FIG. 1) and is customizable to the shape of patient-specific intrauterine anatomy. The radioactive source 20 is positioned in the device 10 after the device 10 is inserted into the patient 2. For example, the secondary radioactive source 20 exits an external machine located next to the patient 2 and travels down the primary applicator 12 after the device 10 is inserted into the patient 2.

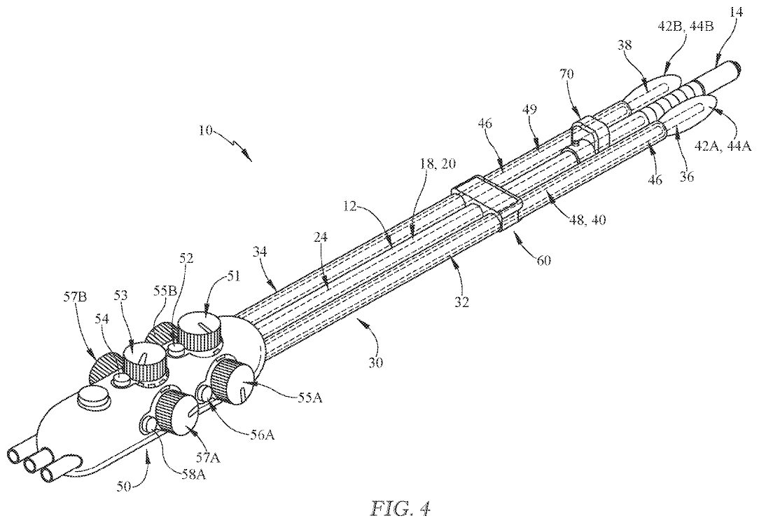

The telescoping primary applicator 14 being customizable to the anatomy of the patient 2 provides a number of potential benefits as described herein. As shown in FIGS. 2 and 3, the telescoping primary applicator 14 includes an extension 22 that holds the radioactive source 20, and primary guide wires 24. The extension 22 is configured to move from a retracted position 25 arranged largely or completely within the primary interior space 18 of the primary guide 12 as shown in FIG. 3 to an extended position 26 arranged largely outside the primary interior space 18 of the primary guide 12 as shown in FIG. 2.

The extension 22 is configured to have a customized arcuate shape when in the extended position 26. As shown in FIGS. 2 and 3, the primary guide wires 24 are coupled to the primary applicator 14 and/or the extension 22 and each extend along a length of the extension 22 at a different location around the extension 22. The radioactive source 20 is temporarily held in a preselected location within the patient 2 using the primary applicator 14.

As shown in FIGS. 2-5, the control system 50, illustratively provided by a rotatable knob 51, is configured to adjust the length of each of the primary guide wires 24 to drive motion of the extension 22 in/out of the primary interior space 18. The control system 50, illustratively provided by a rotatable knob 52, is configured to lock the length of the primary applicator 14 and/or the extension 22. Furthermore, the control system 50, illustratively provided by a rotatable knob 53. is configured to bend the extension 22 and select the customized arcuate shape of the extension 22 so that the overall shape of the device 10 corresponds to the shape the patient 2. The control system 50 is configured to bend the extension 22 at an angle relative to the primary guide axis 16 to form the customized arcuate shape. The control system 50, illustratively provided by a rotatable knob 54, is configured to lock the angle of the extension 22.

In some embodiments, the control system 50 is configured to bend the extension 22 at an angle relative to the primary guide axis 16 to form the customized arcuate shape before the control system 50, is configured to drive motion of the extension 22. In other embodiments, the control system 50 is configured to bend the extension 22 at an angle relative to the primary guide axis 16 to form the customized arcuate shape after the control system 50, is configured to drive motion of the extension 22. The extension 22 comprises a bendable, flexible material

As shown in FIG. 4, the locator assembly 30 includes two secondary guides 32, 34 and two secondary applicators 36, 38. In some embodiments, the locator assembly 30 may include only one secondary guide 32 or 34 and one secondary applicator 36 or 38. Each secondary applicator 36, 38 is configured to house a secondary radioactive source 40. The secondary radioactive source 40 is positioned in the locator assembly 30 of the device 10 after the device 10 is inserted into the patient 2. For example, the secondary radioactive source 40 source exits an external machine located next to the patient 2 and travels down the secondary applicators 36, 38 after the device 10 is inserted into the patient 2.

Each secondary guide 36, 38 is coupled or connected to an expandable structure 42A, 42B and a flexible skin 44A, 44B. The flexible skin 44A, 44B is configured to prevent the secondary radioactive source 40 from contacting patient tissue. In some embodiments, the flexible skin 44A, 44B may comprise a flexible material (e.g., silicon).

The primary guide 12 and the two secondary guides 32, 34 are held together by a locking grip 60. Each secondary guide 32, 34 can be attached or detached to the primary guide 12 by the locking grip 60 before and/or after insertion into the patient 2. The primary guide includes a flange 70 that can prevent the extension 22 from dislodging after the primary applicator 14 and/or the extension 22 is customized to the shape of patient-specific intrauterine anatomy.

In the illustrated example, shown in FIG. 4, each of the secondary guide 32, 34 is a hollow tube 32. 34. The expandable structure 42A, 42B is configured to engage the patient 2 and hold the device 10 in place during a procedure. The secondary radioactive source 40 travels through each of the secondary applicator 36. 38 in the secondary guides 32, 34. In some embodiments, the secondary applicator 36, 38 remains in the extended position outside the secondary guide 32, 34. In other embodiments, the secondary applicator guides 36, 38 may be retracted into the secondary guide 32, 34 before insertion and/or removal from the patient 2. The secondary applicator 32. 34 may further comprises secondary guide wires 46 configured to move the secondary radioactive source 40 from a retracted position inside a secondary interior space 48 inside the secondary guide 32, 34 to an extended position outside the secondary interior space 48.

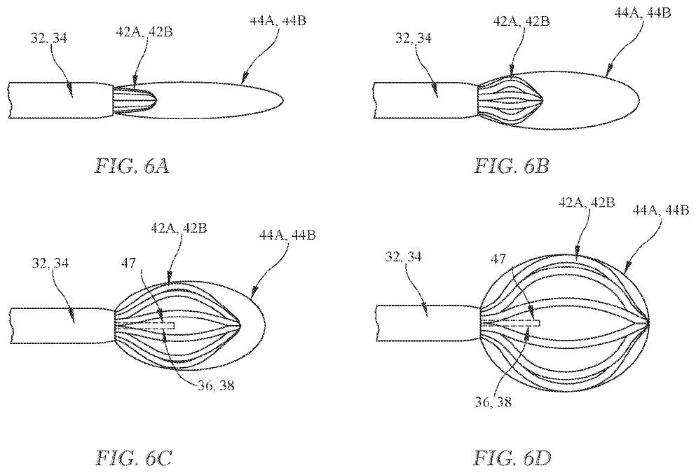

As shown in FIGS. 6A-6D, the expandable structure 42A, 42B is a collapsible structure 42A, 42B. The expandable structure 42A, 42B can expand or inflate to form a sphere. FIGS. 6A-6D illustrate different stages in the inflation of the expandable structure 42A, 42B. The expandable structure 42A, 42B can collapse before removal from the patient 2. FIG. 6A illustrates a completely collapsed expandable structure 42A, 42B. FIG. 6D illustrates a completely expanded expandable structure 42A, 42B. FIGS. 6B and 6C illustrate the expandable structure 42A, 42B comprising a diameter larger than the completely collapsed diameter and smaller than the completely expanded diameter.

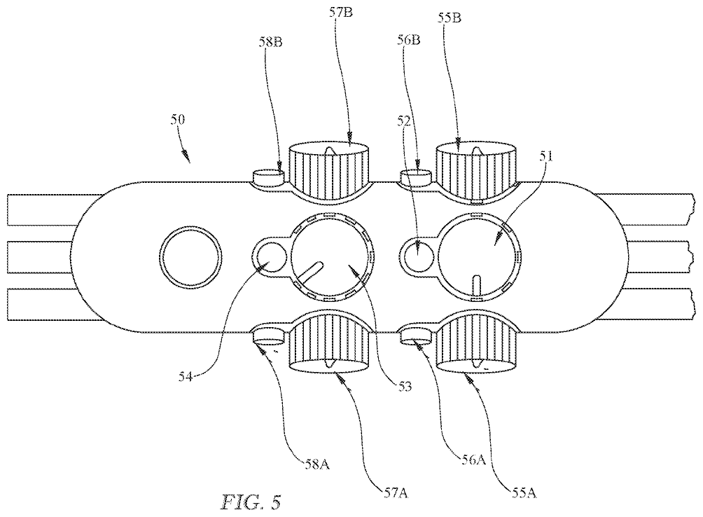

In other embodiments, the expandable structure 42A, 42B can expand or inflate to form a shape different from a sphere. The control system 50, illustratively provided by rotatable knobs 55A, 55B, is configured to separately manage the expansion of each expandable structure 42A, 42B. The control system 50, illustratively provided by rotatable knobs 56A, 56B (see FIG. 5), is configured to separately lock the expansion of each expandable structure 42A, 42B. In some embodiments, change in the diameter of the expandable structure 42A, 42B results in an automatic change in position of the secondary radioactive source 40 such that a free end 47 of the secondary applicator 36. 38 is maintained at a center of the expandable structure 42A, 42B (see FIGS. 6D).

As shown in FIG. 4, each secondary guide 32, 34 is configured to include lateral guide wires 49 that enable lateral movement of the secondary guide 32, 34 after insertion. The lateral movement of the secondary guide 32, 34 may be away or toward the primary guide 12. The control system 50, illustratively provided by rotatable knobs 57A, 57B is configured to separately manage the lateral movement of each secondary guide 32, 34. In some embodiments, the lateral movement of each secondary guide 32, 34 is dependent on the position of the associated expandable structure 42A, 42B such that the expandable structure 42 does not touch the primary applicator 12 when the expanded structure 42A, 42B is in the completely expanded position. The control system 50, illustratively provided by rotatable knobs 58A, 58B (see FIG. 5), is configured to separately lock each lateral guide wire 49 after lateral movement of the secondary guide 32, 34. As shown in FIG. 5, the control system 30 includes a device lock 72 that is configured to lock all knobs 51, 53, 55. 57A, 57B. The knobs 51, 53, 55, 57A, 57B are configured to move their respective components in an incremental manner.

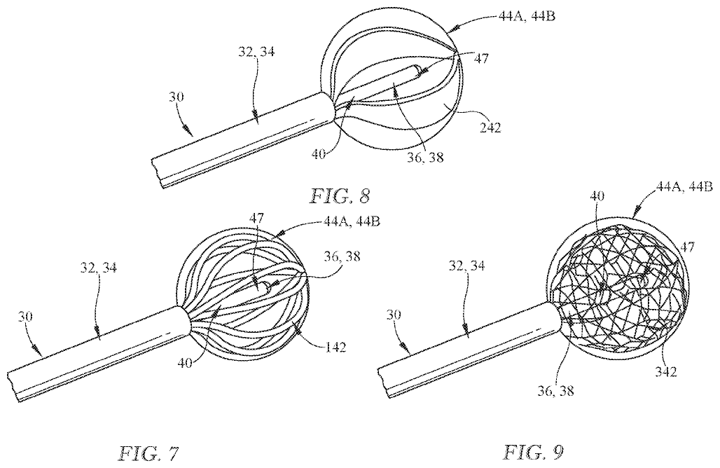

In one embodiment of the present disclosure, as shown in FIG. 7, the expandable structure is a blossom structure 142 that can be expanded into a sphere comprising a diameter that ranges from about 1 cm to about 3 cm including any diameter of range comprised therein. The blossom structure 142 is made of titanium or other flexible yet rigid. MRI safe material. In some embodiments, blossom structure 142 can be expanded into a sphere comprising a diameter less than about 1 cm or more than about 3 cm. The secondary applicator 36, 38 is surrounded by the blossom structure 142. The free end 47 of the secondary applicator 36, 38 is positioned at the center of the blossom structure 142. In other embodiments, the free end 47 of the secondary applicator 36, 38 may not be positioned at the center of the blossom structure 142.

In another embodiment of the present disclosure, as shown in FIG. 8, a sail or ribbon concept comprising a ribbon structure 242 is used. The locator assembly 30 of the ribbon structure 242 includes the secondary applicator 36, 38 through which the second radioactive 40 source travels. The ribbon structure 242 may be easier to insert into a patient 2 in the collapsed configuration. The ribbon structure 242 is an expandable sheet made of titanium or other flexible yet rigid, MRI safe material. The ribbon structure 242 will be inserted into the vagina 4 directly beneath the cervix 6 (one sail on either side of the cervix 6) and then expanded. After radioactive therapy delivery, the ribbon structure 242 will be collapsed prior to removal of the device 10.

In another exemplary embodiment of the present disclosure, a mesh concept comprising a mesh structure 342 is used as shown in FIG. 9. The locator assembly 30 of the mesh concept 342 includes the secondary applicators 36, 38 through which the second radioactive 40 source travels. The mesh structure 342 may provide additional mechanical support to prevent the collapse of the flexible skin 44A, 44B after inflation. The mesh structure 342 is an expandable sheet made of titanium or other flexible yet rigid, MRI safe material. The mesh structure 342 will be inserted into a vagina 4 directly beneath cervix 6 (one sail on either side of the cervix) and then expanded. After radioactive therapy delivery, the mesh structure 342 will be collapsed prior to removal of the device 10. In some embodiments, the device 10 may include an air hose fluidly coupled to the expandable structure 42A, 42B shown in FIGS. 6A-6D to carry air into and out of the expandable structure 42A, 42B to control the diameter of the expandable structure 42A, 42B.

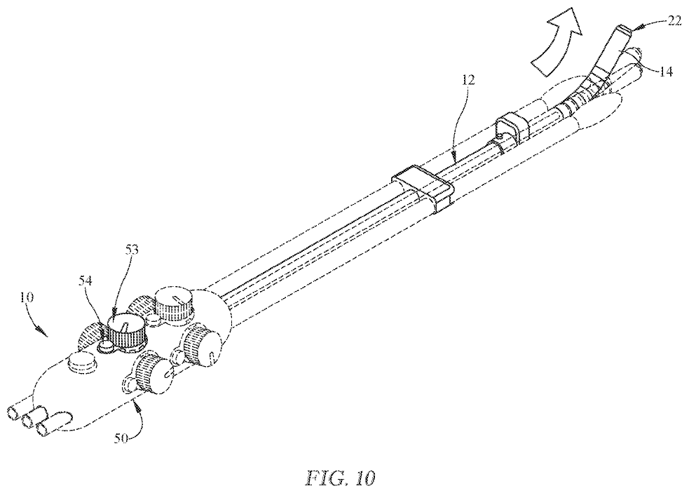

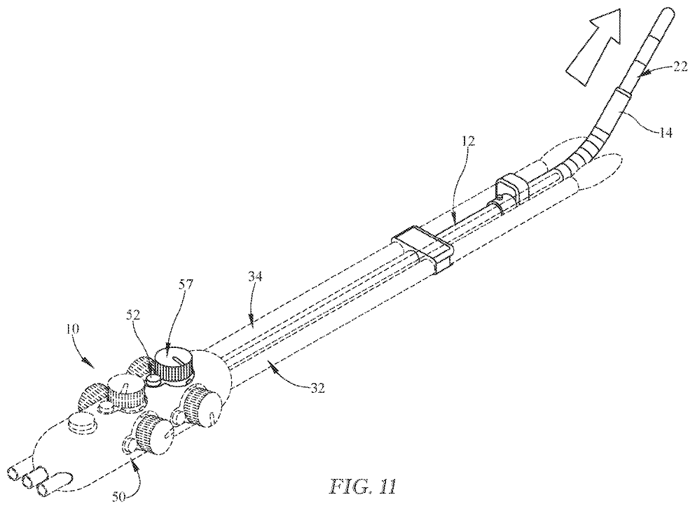

One embodiment of the present disclosure describes a method 200 of performing an intrauterine brachytherapy procedure as shown in FIGS. 10-20, and 23. As shown in FIG. 1 the device 10 is inserted into the vagina 4, directly beneath the cervix 6. After radioactive therapy delivery, the expandable structure 42A, 42B shown in FIG. 4 will be collapsed or deflated prior to removal of the device 10.

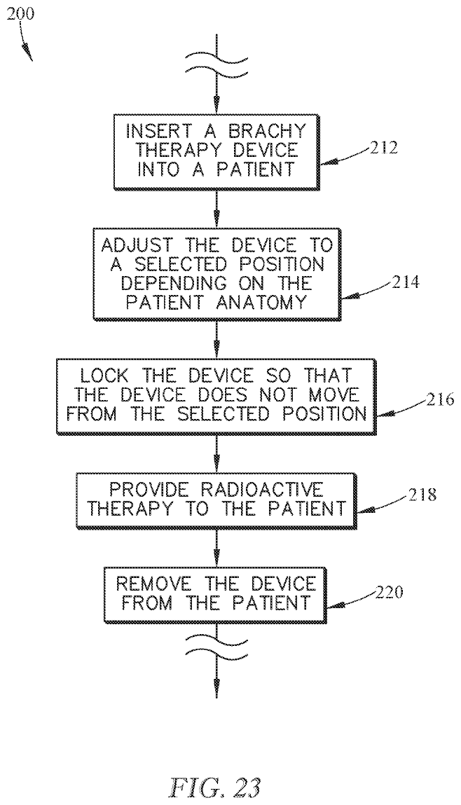

The method includes inserting the brachytherapy device 10 into the patient 2 as shown in step 212 (FIG. 23), adjusting the brachytherapy device 10 to a selected position depending on the patient anatomy as shown in step 214 (FIG. 23), locking the brachytherapy device 10 so that the device 10 does not move from the selected position as shown in step 216 (FIG. 23), providing radioactive therapy to the patient 2 by using the brachytherapy device 10 as shown in step 218 (FIG. 23), and removing the brachytherapy device 10 from the patient 2 as shown in step 220 (FIG. 23).

As shown in FIGS. 1, 4, and 23, the method includes inserting the brachythcrapy device 10 including the primary guide 12 and two secondary guides 32, 34 simultaneously into the patient 2. In other embodiments, the method may include inserting the primary guide 12 and two secondary guides 32, 34 sequentially into the patient 2. The method further includes using the knob 53 to bend the extension 22 to select the angle for customizing the arcuate shape of the extension 22 so that the overall shape of the device 10 corresponds to the shape of the patient 2 (see FIG. 10). The knob 54 is used to lock the angle of the extension 22.

The method further includes using the knob 51 to manage the length of the primary applicator 14 and/or the extension 22 to select the customized arcuate shape of the extension 22 so that the overall shape of the device 10 corresponds to the shape the patient 2 (see FIG. 10). The knob 52 is used to lock the length of the primary applicator 14 and/or the extension 22.

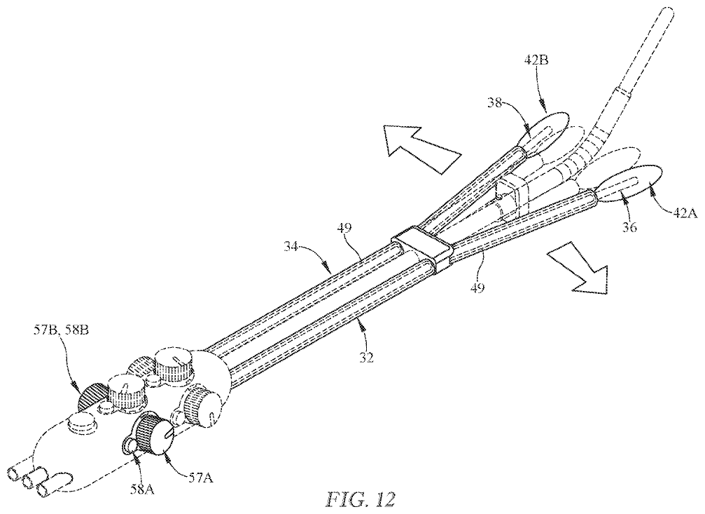

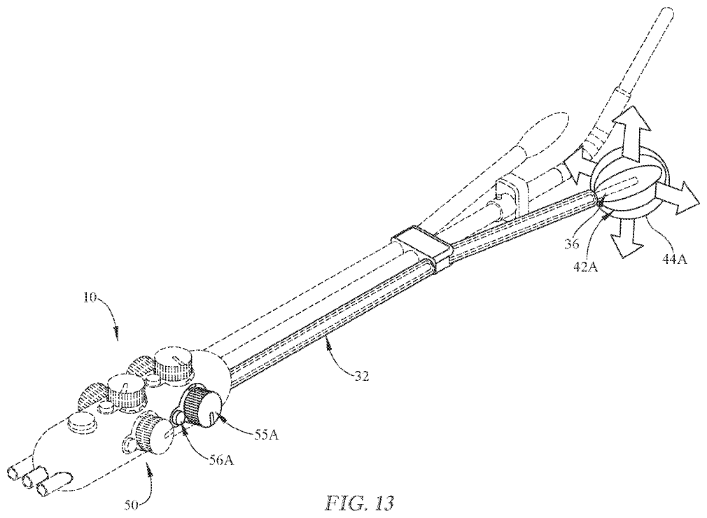

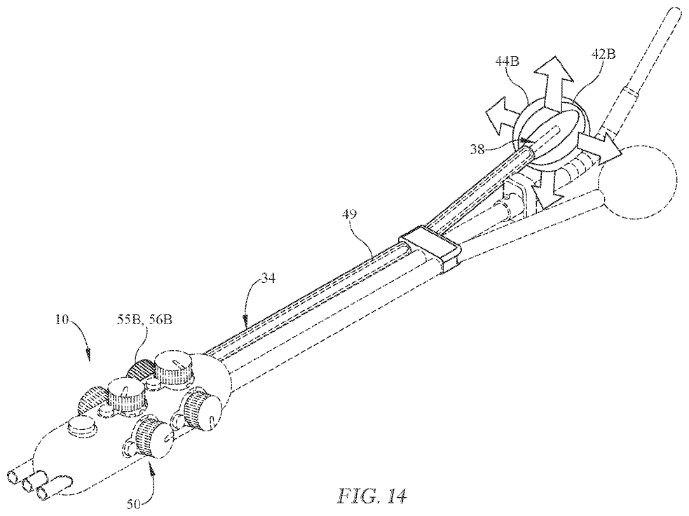

As shown in FIG. 12, the method further includes using the knobs 57A, 57B to manage the lateral movement of the secondary guides 32, 34. The knobs 58A, 58B (see FIG. 5) is used to lock the lateral position of each secondary guide 32. 34 by adjusting the lateral guide wires 49. As shown in FIG. 13, the method further includes using the knob 55A to manage the expansion of the first expandable structure 42A. The knob 56A is used to lock the expansion of the first expandable structure 42A. As shown in FIG. 14, the method further includes using the knob 55B to manage the expansion of the second expandable structure 42B. The knob 56B (see FIG. 5) is used to lock the expansion of the second expandable structure 42B.

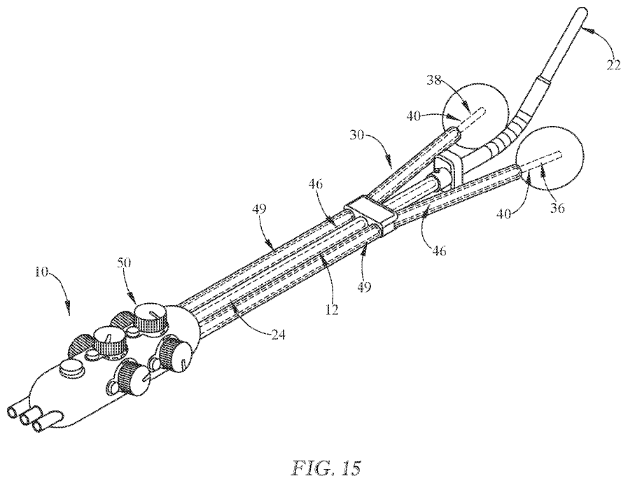

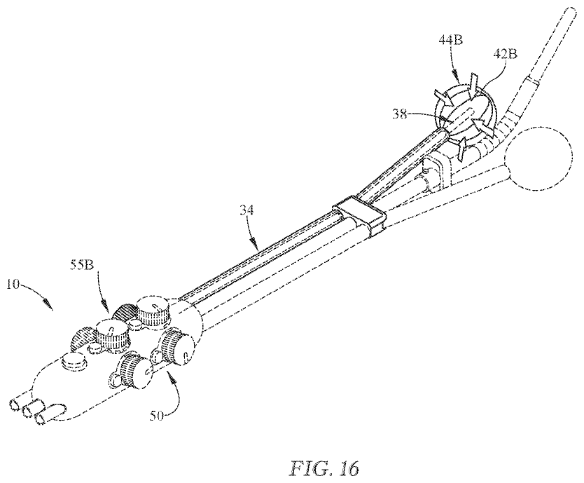

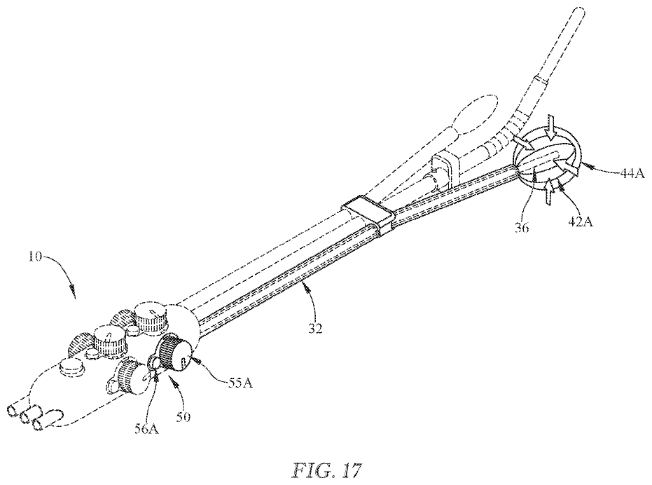

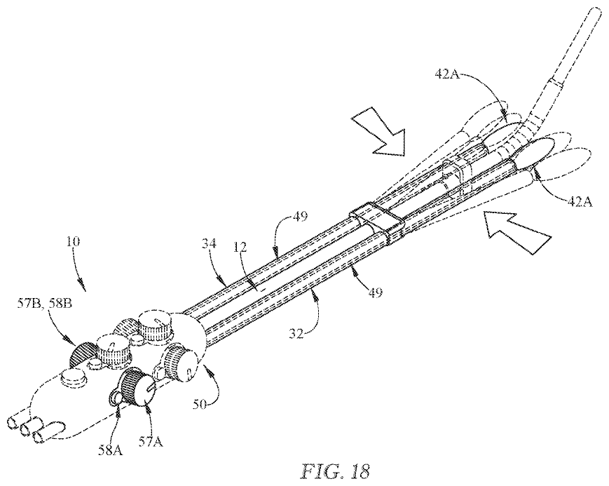

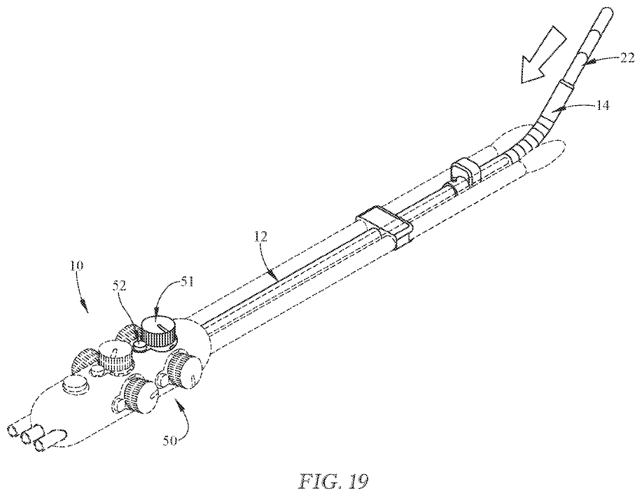

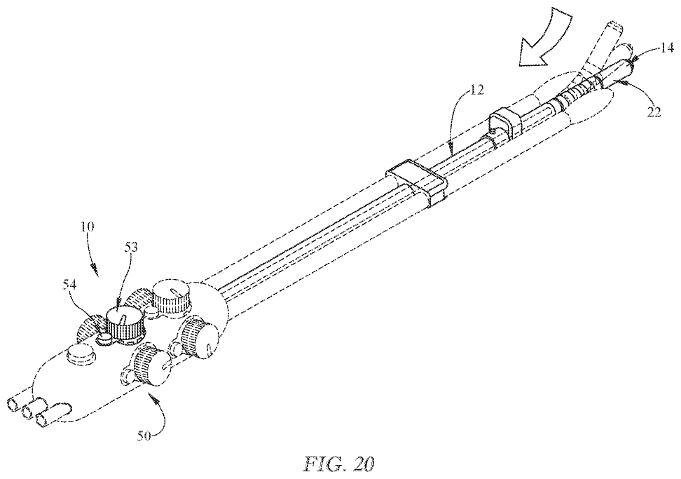

As shown in FIG. 15, the method further includes using the device 10 to provide radioactive therapy to the patient 2. After providing radioactive therapy, as shown in FIG. 16, the method includes using the knob 55B to retract the expansion of the first expandable structure 42B. As shown in FIG. 17, the method includes using the knob 55A to retract the expansion of the second expandable structure 42A. As shown in FIG. 18. the method includes using the knobs 57A, 57B to manage the lateral movement of the secondary guides 32, 34. The secondary guides 32, 34 are moved towards the primary guide 12. As shown in FIG. 19, the method includes using the knob 51 to retract the extension 22. As shown in FIG. 20, the method includes using the knob 53 to manage the angle the extension 22. Changing the length and the angle of the primary applicator 14 and/or the extension increases the ease of removing the device 10 from the patient 2.

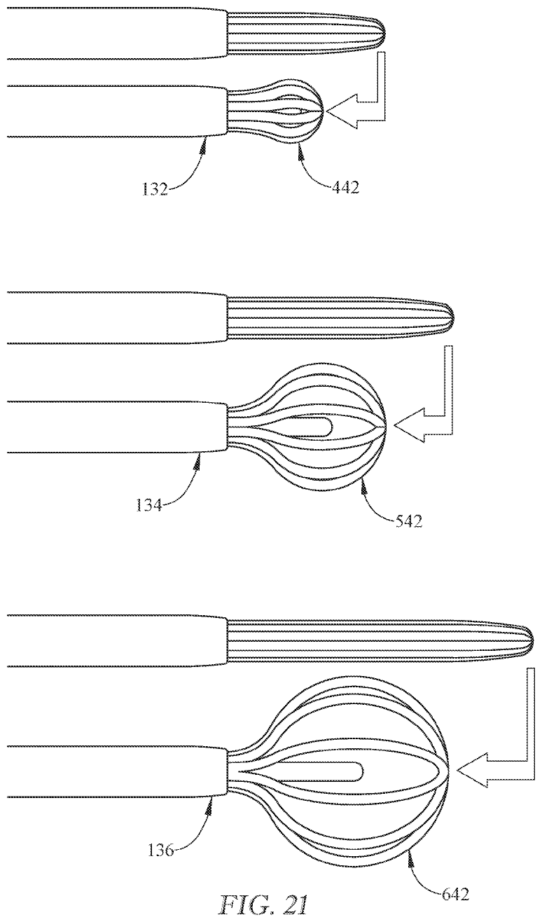

In one embodiment described herein is a kit comprising different components of a brachytherapy device 10. The kit includes the primary guide 12 and/or one or more secondary guides 132, 134, 136 with expandable structures 442, 542, 642 (shown in FIG. 21. The expandable structures 442, 542, 642 have varying maximum diameters upon inflation. For example, the expandable structures 442, has a maximum inflated diameter of 1 cm, the expandable structures 542, has a maximum inflated diameter of 2 cm, and the expandable structures 642, has a maximum inflated diameter of 3 cm. In other embodiments, the maximum inflated diameter of the expandable structure 442, 542, 642 may range from about 0.5 cm to about 4 cm, including any diameter or range comprised therein.

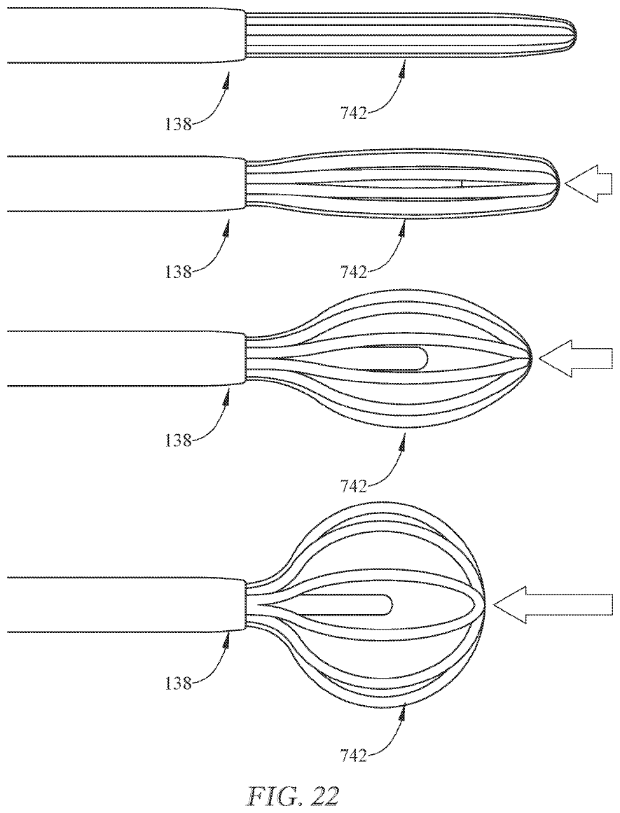

In some embodiments, as shown in FIG. 22, a secondary guide 138 has an adjustable expandable structure 742. The expandable structures 742 can be adjusted and locked at varying diameters depending on patient-specific intrauterine anatomy. In some embodiments, the kit may include one or more secondary guides with adjustable expandable structures 742 and one or more secondary guides with fixed expandable structure 442, 542, 642.

In some embodiments, the kit may include one to ten secondary guides 132, 134, 136, 138, and/or one to four primary guides 12. In some embodiments, the kit may include secondary guides 132, 134, 136, 138 with the blossom structure 142, the ribbon structure 242, and/or the mesh structure 342. In some embodiments, the kit may include more than one the primary guide 12. In some embodiments, the kit may include one or more secondary guides 132, 134, 136, 138 and no primary guides.

Those skilled in the art will understand that the devices and methods specifically described herein and illustrated in the accompanying drawings are non-limiting examples. The features illustrated or described in connection with one exemplary embodiment may be combined with the features of other embodiments. Such modifications and variations arc intended to be included within the scope of the present disclosure. Accordingly, aspects and features of every embodiment may not be described with respect to each embodiment, but those aspects and features are applicable to the various embodiments unless statements or understandings are to the contrary.

The figures provided herein are not necessarily to scale, although a person skilled in the art will recognize instances where the figures are to scale and/or what a typical size is when the drawings are not to scale. Additionally, a number of terms may be used throughout the disclosure interchangeably but will be understood by a person skilled in the art. Further, to the extent features, sides, or steps are described as being “first” or “second,” such numerical ordering is generally arbitrary, and thus such numbering can be interchangeable. Lastly, the present disclosure includes some illustrations and descriptions that include prototypes or bench models. A person skilled in the art will recognize how to rely upon the present disclosure to integrate the techniques, systems, devices, and methods provided for into a product in view of the present disclosures.

1. A brachytherapy device for administration of intrauterine radiation, the device comprising
a primary guide configured to be inserted into a patient that extends along a primary guide axis and that defines a primary interior space along the primary guide axis,
a telescoping primary applicator configured to temporarily house a first radioactive source within the patient that is customizable to the shape of patient-specific intrauterine anatomy, the telescoping primary applicator including
(i) an extension configured to move from a retracted position arranged within the primary interior space of the primary guide to an extended position arranged largely outside the primary interior space of the primary guide, wherein the extension is configured to have a customized arcuate shape when in the extended position, and
a locator assembly comprising
(i) at least one secondary guide configured to be inserted into the patient that extends along a secondary guide axis and that defines a secondary interior space along the secondary guide axis, the secondary guide further including
(i) an expandable structure surrounding the secondary radioactive source, the expandable structure configured to be in a position that ranges from a completely expanded position to a completely collapsed position, and
(ii) a flexible skin configured to cover the expandable structure, and
(ii) a secondary applicator positioned in each secondary guide configured to temporarily house a secondary radioactive source in the secondary interior space, and
a control system including a first user input configured to drive motion of the extension in/out of the primary interior space and a second user input configured to control the position of the expandable structure.

2. The device of claim 1, wherein the primary applicator includes primary applicator guide wires coupled to the extension that each extend along a length of the extension at a different location around the extension and the first knob is configured to adjust the length of each of the primary applicator guide wires to drive the motion of the extension in/out of the primary interior space

3. The device of claim 1, wherein the control system further includes a third knob configured to lock the length of the extension and a fourth knob configured to lock the position of the expandable structure.

4. The device of claim 1, wherein when the telescoping primary applicator is configured to bend at an angle relative to the primary guide axis to form the customized arcuate shape before the extension is configured to move from the retracted position to the extended position, and the control system further includes a third knob configured to control the angle of the extension and a fourth knob configured to lock the angle of the extension.

5. The device of claim 1, wherein the secondary applicator further comprises
secondary applicator guide wires configured to move the secondary radioactive source from a retracted position inside the secondary interior space to an extended position outside the secondary interior space,
the expandable structure is configured to surround the secondary radioactive source when the secondary radioactive source is in the extended position outside the secondary interior space, and
the control system further includes a third user input configured to adjust a length of each of the secondary applicator guide wires to drive motion of the secondary radioactive source in/out of the secondary interior space and a fourth user input configured to lock the length of each of the secondary applicator guide wires.

6. The device of claim 5, wherein the secondary applicator guide wires are configured to position the second radioactive source at more than one extension point when the secondary radioactive source is in the extended position outside the hollow tube.

7. The device of claim 1, wherein the device comprises at least two secondary applicators.

8. The device of claim 1, wherein the locator assembly is configured to substantially fix the telescoping primary applicator relative to the patient prior to moving the extension from the retracted position during a brachytherapy procedure.

9. The device of claim 1, wherein the expandable structure includes at least one of (a) a blossom provided by a wireframe configured to be expanded and contracted, (b) a sail provided by a sheet material configured to be expanded and contracted, and (d) a wire mesh configured to be expanded and contracted.

10. (canceled)

11. (canceled)

12. The device of claim 1, wherein the primary guide is a hollow tube that defines the primary interior space.

13. The device of claim 1, wherein the secondary guide is a hollow tube that defines the secondary interior space.

14. The device of claim 1, wherein the secondary guide includes secondary guide wires to aid in a lateral movement of the secondary guide relative to the primary guide from a first position to a second position, and the control system further includes a third user input configured to control the lateral movement of the secondary guide and a fourth user input to lock the secondary guide in the second position.

15. The device of claim 1, wherein the lateral movement of the secondary guide is dependent on the position of the expandable structure such that the expandable structure does not touch the primary applicator when the expanded structure is in the completely expanded position.

16. The device of claim 1, wherein the primary guide and the secondary guide are connected by a locking mechanism.

17. The device of claim 1, wherein the primary guide includes a flange that can prevent the extension from dislodging after the extension is customized to the shape of patient-specific intrauterine anatomy.

18. The device of claim 1, wherein the control system includes a device lock configured to lock all components of the device.

19. The device of claim 1, wherein the primary applicator includes primary applicator guide wires coupled to the extension and the first knob is graded to identify the length of each of the primary applicator guide wires and the second knob is graded to identify the position of the expandable structure.

20. (canceled)

21. (canceled)

22. A brachytherapy device for administration of intrauterine radiation, the device comprising
at least one guide configured to be inserted into a patient that extends along a guide axis and that defines an interior space along the guide axis,
an applicator positioned in each guide configured to temporarily house a radioactive source, the guide including
i. an expandable structure surrounding the radioactive source configured to comprise an expanded diameter or a collapsed diameter, and
ii. a flexible skin configured to cover the expandable structure, and
a control system including a first user input configured to control the diameter of the expandable structure and a second user input configured to lock the diameter of the expandable structure, wherein the flexible skin is configured to prevent the radioactive source from contacting patient tissue.

23. (canceled)

24. The device of claim 22, wherein the applicator further comprises guide wires on the applicator configured to move the radioactive source from a retracted position inside the interior space to an extended position outside the interior space,
the expandable structure is configured to surround the radioactive source when the radioactive source is in the extended position outside the interior space, and
the control system further includes a third user input configured to adjust a length of each of the guide wires to drive motion of the secondary radioactive source in/out of the secondary interior space and a fourth user input configured to lock the length of the guide wires.

25. (canceled)

26. (canceled)

27. (canceled)

28. (canceled)

29. (canceled)

30. The device of claim 22, wherein the guide is a hollow tube that defines the interior space.

31.-62. (canceled)
